(19) United States
(12) Patent Application Publication
(10) Pub. No.: US 2004/0228847 A1
(43) Pub. Date: Nov. 18, 2004

(54) Progenitor cells and methods of using same

(75) Inventors: Pascal J. Goldschmidt-Clermont, Chapel Hill, NC (US); Doris A. Taylor, Saint Paul, MN (US); Frederick M. Rauscher, Miami, FL (US); Robert Judd, Chapel Hill, NC (US); Raymond Kim, Chapel Hill, NC (US)

Correspondence Address: NIXON & VANDERHYE, PC, 1100 N GLEBE ROAD, 8TH FLOOR, ARLINGTON, VA, 22201-4714, US

(73) Assignee: DUKE UNIVERSITY, Durham, NC

(21) Appl. No.: 10/788,423

(22) Filed: Mar. 1, 2004

Related U.S. Application Data
(60) Provisional application No. 60/450,340, filed on Feb. 28, 2003.
(60) Provisional application No. 60/474,236, filed on May 30, 2003.

(51) Int. Cl. A61K 48/00

(52) U.S. Cl. 424/93.21; 424/93.71

(57) ABSTRACT

The present invention relates generally to stem/progenitor cells and, in particular, to therapeutic strategies based on the use of such cells to effect vascular rejuvenation and/or to serve as delivery vehicles.

This application claims priority from U.S. Provisional Application No. 60/450,340, filed Feb. 28, 2003, and from U.S. Provisional Application No. 60/474,236, filed May 30, 2003, the entire disclosures of both provisional applications being incorporated herein by reference.

### TECHNICAL FIELD

The present invention relates generally to stem/progenitor cells and, in particular, to therapeutic strategies based on the use of such cells to effect vascular rejuvenation and/or to serve as delivery vehicles.

### BACKGROUND

Chronic vascular injury, in the form of mechanical stress and excess cholesterol, is believed to cause atherosclerosis. However, concomitant vascular aging, the mechanism of impact of which remains unaccounted for, represents a profound risk factor for atherosclerosis. While young arteries are remarkably resistant to vascular injury, aging arterial vessels display increased turnover of their constituent cells, accompanied by a switch to senescent, dysfunctional and pro-inflammatory phenotypes (Ross, Nature 362:801 (1993), Chang et al, Proc. Natl. Acad. Sci. USA 92:11190 (1995), Okuda et al, Atherosclerosis 152:391 (2001)).

The present invention results, at least in part, from the realization that the switch to senescent, dysfunctional and pro-inflammatory phenotypes is a critical determinant of atherosclerotic progression and results from a progressively inadequate number of vascular progenitor cells required for the repair of damaged blood vessels.

The invention provides a method of inhibiting progression of atherosclerosis that utilizes stem cell or vascular progenitor cell transplantation. The invention further provides a method of delivering agents, including therapeutic and imaging agents, to vessel walls using stem/progenitor cells as carriers.

### SUMMARY OF THE INVENTION

The present invention relates to therapeutic strategies based on the use of progenitor (precursor) cells (or stem cells) to effect vascular rejuvenation and/or to serve as delivery vehicles.

Objects and advantages of the present invention will be clear from the description that follows.

### BRIEF DESCRIPTION OF THE DRAWINGS

FIGS. 1A-1G. Atherosclerosis assessment in untreated (FIGS. 1A-1C) and bone marrow (BM)-treated (FIGS. 1D-1F) ApoE^{-/-} mice. FIGS. 1A and 1D, Gross visualization of aortic arch. FIGS. 1B and 1E, Cross sections of innominate artery. FIGS. 1C and 1F, Oil red O-stained proximal aortic root. FIG. 1G, All atherosclerosis data (mean±SEM) are for ApoE^{-/-} “recipient” mice maintained on high-fat diet, sorted into 1 of 6 groups (a-f), at 14 weeks of age. Cell injections were given at 2-week intervals from 3 weeks until 13 weeks of age (1×10⁶ cells/injection). Groups a, b, e, and f received cells intravenously. Donor cells originated from severely atherosclerotic 6-month-old ApoE^{-/-} mice, maintained on high-fat diet (a), preatherosclerotic 4-week-old ApoE^{-/-} mice (b), or nonatherosclerotic WT mice on normal chow diet (e and f). Group c indicates mice given no cells (negative control). Group d received WT cells intraperitoneally (“cell-positive” negative control). Groups a, b, and d received combined stromal- and hematopoietic-enriched cells. Groups a and b differed from each other only in age of cell donor. *Atherosclerotic burden differs significantly between groups a and b at each anatomic location indicated (P<0.05). **Atherosclerosis burden calculations differ by anatomic location.

FIGS. 2A-2C. Age-related CD31+/CD45− cell loss in ApoE^{-/-} mice. BM was obtained from 6-month-old WT mice on chow diet, 6-month-old ApoE^{-/-} mice on high-fat diet, and 1-month-old ApoE^{-/-} mice. Hematopoietic-enriched cells from each mouse (50 000 total; n=5 to 6/group) were sorted by FACS. FIG. 2A, Characteristic front scatter/sidescatter (FSC/SSC) plot shows significant decrease in cell numbers at left lower corner (red circle) in old ApoE^{-/-} mice. In contrast, these cells were enriched in young ApoE^{-/-} mice. FIG. 2B, Back-tracing of encircled cell population. CD31+/CD45− cells appear blue, CD31+/CD45+ cells appear red, and CD31− cells appear gray. Clear colocalization is observed between missing cells (within red circle) in FIG. 2A and blue cells in FIG. 2B. FIG. 2C, Dual-channel flow cytometry analysis of CD31 and CD45 identified this subpopulation as being CD31+/CD45−, a characteristic feature of endothelial progenitor cells. Boxed numerals indicate percent of cells gated for each quadrant for this representative trial.

FIGS. 3A-3G. β-Gal-positive donor cell localization. Combined hematopoietic- and stromal-enriched BM cells from donor mice that expressed β-gal were injected into ApoE^{-/-} recipients on high-fat diet (n=4) or WT recipients on normal chow diet (n=4) (1×10⁶ cells/injection every 2 weeks for 3 injections, beginning at 4 weeks of age). FIGS. 3A-3E, Whole aortas opened lengthwise and stained en face. β-Gal-positive cells (blue) localize to most atherosclerosis-prone regions of aorta in ApoE^{-/-} mice (FIG. 3A). There is no β-gal staining in untreated ApoE^{-/-} mice (FIG. 3B) and very little in BM-treated WT mice (FIG. 3C). Oil red O staining reveals much less lipid deposition in BM-treated ApoE^{-/-} mice (FIG. 3D) than in untreated mice (FIG. 3E), particularly in regions of aorta with most donor cell localization (FIGS. 3A and 3E). FIGS. 3F and 3G, Frozen sections of aortas from BM-treated ApoE^{-/-} mice showing vascular engraftment of donor cells. β-Gal-positive donor cells (FIG. 3F, blue) also stained positively for CD31 (FIG. 3G, red), an endothelial cell marker (arrows).

FIGS. 4A-4E. Suppression of IL-6 by BM cell injection. FIGS. 4A-4D, Six-month-old ApoE^{-/-} mice were injected intravenously with 2×10⁶ combined hematopoietic- and stromal-enriched cells from 6-month-old WT or 6-month-old ApoE^{-/-}. Donors were maintained on either regular (R) or fat-rich (F) diets. For each donor type, 7 to 8 recipients were treated, and blood was drawn for analysis 15 days after cell injection. FIGS. 4A and 4B, Plasma cholesterol levels in untreated mice (FIG. 4A) and ApoE^{-/-} mice treated with BM from WT and ApoE^{-/-} mice (FIG. 4B). FIGS. 4C and 4D, Plasma IL-6 levels in untreated mice (FIG. 4C) and in ApoE^{-/-} mice treated with BM from WT and ApoE^{-/-} mice (FIG. 4D). FIG. 4E, Six-month-old ApoE^{-/-} mice were injected intravenously with 2×10⁶ hematopoietic-enriched cells from 6-month-old WT, 6-month-old ApoE^{-/-}, or 4-week-old ApoE^{-/-} donors. Donors were maintained on either regular (R) or fat-rich (F) diets, and 7 to 8 recipients were treated for each donor type. Plasma IL-6 levels were measured at 0, 15, or 30 days after cell injection. *P<0.05, **P<0.01, ***P<0.001 compared with control (leftmost bar on each graph). †P<0.05 compared with WT donors on regular or fat-rich diet.

FIG. 5. Telomere length assay on aortic intimal cells from untreated ApoE^{-/-} mice (lanes 1 to 4), BM-treated ApoE^{-/-} mice (1×10⁶ WT cells/injection, combined hematopoietic- and stromal-enriched cells, every 2 weeks for six injections; lanes 5 to 10), and congenic, untreated, nonatherosclerotic WT mice (lanes 11 and 12). BM-treated mice had significantly longer telomeres than untreated mice, indicating attenuated vascular senescence.

FIG. 6. Atherosclerosis assessment in recipients of ApoE^{-/-} BM (n=6/group). Anti-atherosclerososis efficacy is dependent on the age and atherosclerotic status of the donor, with greater efficacy of vascular progenitor cells from young, pre-atherosclerotic mice.

FIGS. 7A-7D. FIGS. 7A-7C. Demonstration of visualization using MRI technology of stem cells that have engulfed nano- and micro-particles of iron. FIG. 7D. Visualization of Feridex loaded stem cells injected into the cardiovascular system.

### DETAILED DESCRIPTION OF THE INVENTION

The present invention is based, at least in part, on the realization that vascular turnover in aging vessels is a critical determinant of initiation and progression of atherosclerosis. Vascular injury (e.g., chemical stress, hemodynamic stress and oxidation/inflammation) leads to turnover of endothelial cells which ultimately leads to atherosclerosis. Because the number of cell divisions is finite, endothelial cell turnover can result in an exhaustion of endothelial repair, which can be a critical time-dependent initiation of atherosclerosis.

The present invention relates to a method of attenuating atherosclerosis progression, even in the continued presence of vascular injury, based on vascular rejuvenation. In accordance with this method, vascular rejuvenation is effected using endothelial/vascular progenitor cell engraftment.

Cells suitable for use in the present invention include endothelial progenitor cells, that is, pluripotent, bipotent or monopotent stem cells capable of maturing at least into mature vascular endothelial cells. Progenitor cells capable of vascular differentiation can be isolated from embryos and from hematopoietic and stromal fractions of bone marrow (BM) (Reyes et al, Blood 98:2615-2625 (2001), Sata et al, Nat. Med. 8:403-409 (2002)). Progenitor cells can also be isolated from peripheral blood (or umbilical cord blood). Advantageously, the cells are derived from young, non-atherosclerotic mammals (e.g., humans).

While unsorted hematopoietic and stromal BM cells were used in the Example that follows, populations of cells significantly enriched in specific cell lineages having a propensity for vascular rejuvenation can also be used. For example, endothelial progenitor cells characterized by highly expressed surface antigens including, for example, one or more vascular endothelial growth factor receptor (VEGFR) (e.g., FLK-1 and FLT-1) can be used, as can endothelial progenitor cells that express the CD34+ marker and/or the AC133 antigen (Yin et al, Blood 90:5002-5112 (1997); Miraglia et al, Blood 90:5013-5021 (1997)). Endothelial progenitor cells suitable for use in the invention can also be characterized by the absence of or lowered expressed of markers such as CD1, CD3, CD8, CD10, CD13, CD14, CD15, CD19, CD20, CD33 and CD41A. Endothelial progenitor cells suitable for use in the invention include, but are not limited to, progenitor cells described in Reyes et al, J. Clin. Invest. 109:337-346 (2002), U.S. Pat. No. 5,980,887 and US patent appliction 20020051762.

Methods of isolating progenitor cells suitable for use in the invention are well known in the art (see, for example, Reyes et al, J. Clin. Invest. 109:337 (2002), Reyes et al, Blood 96:2615-2625 (2001), Sata et al, Nat. Med. 8:403-409 (2002), U.S. Pat. No. 5,980,887 and U.S. patent application 20020051762).

Autologous or heterologous endothelial progenitor cells can be used in accordance with the invention and can be expanded in vivo or ex vivo prior to administration. Expansion can be effected using standard techniques (see, for example, U.S. Pat. No. 5,541,103).

Endothelial progenitor cells can be administered using any of a variety of means that result in vascular distribution (e.g., via catheter or via injection), injection of the cells intravenously being preferred. The optimum number of cells to be administered and dosing regimen can be readily determined by one skilled in the art and can vary with the progenitor cells used, the patient status and the effect sought.

In accordance with the invention, endothelial progenitor cell engraftment can be used prophylactically or therapeutically alone or in combination with other approaches designed to prevent atherosclerosis or to attenuate atherosclerotic progression. In this regard, the progenitor cells of the invention can be manipulated (e.g., prior to administration) to serve as carrier or delivery vehicles of agents that have a therapeutic (e.g., anti-atherosclerotic) effect. Such agents can be proteinaceous or non proteinaceous.

For example, the progenitor cells can be used as vehicles for gene delivery. In accordance with this aspect of the invention, a recombinant molecule comprising a nucleic acid sequence encoding a desired protein, operably linked to a promoter, can be delivered to a vascular site (e.g., an atherosclerotic site). The recombinant molecule can be introduced into the progenitor cells using any of a variety of methods known in the art. An effective amount of the transformed progenitor cells can then be administered under conditions such that vascular distribution is effected, expression of the nucleic acid sequence occurs and production of the protein product results. The recombinant molecule used will depend on the nature of the gene therapy to be effected. Vectors suitable for use in endothelial progenitor cells are well known in the art, as are methods of introducing same into progenitor cells (see, for example, U.S. patent application 20020037278). Promoters can be selected so as to allow expression of the coding sequence to be controlled endogenously (e.g., by using promoters that are responsive to physiological signals) or exogenously (e.g., by using promoters that are responsive to the presence of one or more pharmaceutical).

Any of a variety of encoding sequences can be used in accordance with this aspect of the invention. The nucleic acid can encode, for example, a product having an anti-atherosclerotic effect. For example, nucleic acids encoding proteins that afford protection from oxidative damage (such as superoxide dismutase (see, for example, U.S. Pat. No. 6,190,658 or glutathione peroxidase (see, for example, U.S. patent application 20010029249)) can be used, as can nucleic acids encoding components in the synthetic pathway to nitric oxide (see, for example U.S. Pat. No. 5,428,070) or nucleic acids encoding agents that modulate Toll-like receptor activity (see, for example, U.S. patent application 20030022302). Nucleic acids encoding proteins that lower total serum cholesterol, such as an apoE polypeptide (see, for example, U.S. patent application 20020123093) can be used, as well as nucleic acids that encode agents that modulate expression of or activity of the products of the fchd531, fchd540, fchd545, fchd602 or fchd605 genes (see U.S. patent application 20020102603). Nucleic acids encoding proteins suitable for use in treating inflammatory diseases can also be used, such as the glycogen synthase kinase 3β protein (see U.S. patent application 20020077293). (See also, for example, U.S. patent application 20010029027, 20010053769, and 20020051762 and U.S. Pat. No. 5,980,887).

The progenitor cells can also be used to administer non proteinaceous drugs to vascular sites. Such drugs can be incorporated into the cells in a vehicle such as a liposome or time released capsule.

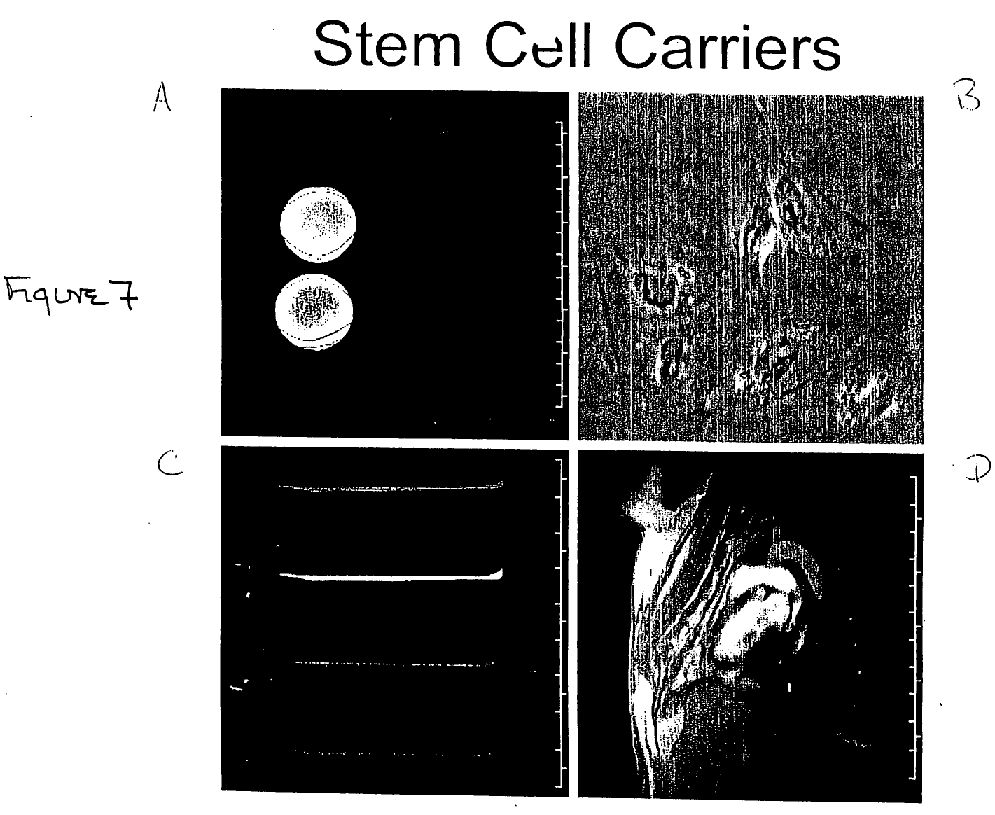

In addition to the use of endothelial progenitor cells as of delivery vehicles for proteinaceous and non-proteinaceous therapeutics, the progenitor cells can also be used to deliver non-therapeutic agents to the vessel wall (see, for example, FIG. 7). Such agents include imaging agents (e.g., MRI imaging agents), such as nano- and micro-particles of iron (e.g., Feridex) and other superparamagnetic contrast agents. The use of such labeled progenitor cells permits monitoring of cellular biodistribution over time. Methods of introducing such agents are known in the art (see, for example, Bulte et al, Nat. Biotechnol. 19:1141-1147 (2001), Lewin et al, Nat. Biotechnol. 18:410-414 (2000), Schoepf et al, BioTechniques 24:642-651 (1998), Yeh et al, Magn. Reson. Imaging 30:617-625 (1997), Lewin et al, Nat. Biotechnol. 18:410-414 (2000), Schoepf et al, BioTechniques 24:642-651 (1998), Yeh et al, Magn. Reson. Imaging 30:617-625 (1997), Frank et al, Acad. Radiol. 9:5484-5487 (2002)). Cell administration methods such as those described above can be used.

As will be appreciated from a reading of this disclosure, the present approaches have applicability in human and non-human animals.

Certain aspects of the invention are be described in greater detail in the non-limiting Example that follows (see also Rauscher et al, Circulation 108:457-463 (2003) and Goldschmidt-Clermont et al, SAGE KE Nov. 12, 2003, pp.1-5 (http://sageke.sciencemag.org/cgi/content/full/sageke;2003/45/re8)).

### EXAMPLE

Experimental Details

Animals. All mice were purchased from Jackson Laboratory (Bar Harbor, Me.). Animals fed a high-fat diet were given diet #88137 (Harlan-Teklad; 42% fat, 1.25% cholesterol) beginning at 3 weeks of age. BM injections were via the internal jugular vein under ketamine anesthesia or via intraperitoneal cavity (controls).

Cells. BM isolated from tibiae and femora was cultured in minimum essential medium alpha (Invitrogen) with 12.5% fetal calf and 12.5% equine serum and 2 μmol/L hydrocortisone. After 2 days, hematopoietic-enriched (nonadherent) cells were suspended in 0.9% NaCl and immediately used for injection. Stromal-enriched (adherent) cells were expanded for 2 weeks before injection.

Pathology. Aortic arches were photographed through a Leica M-650 microscope. Whole aortas, opened lengthwise, and microscopic frozen sections of aortic root were stained with oil red O and quantified. Means and SEMs for atherosclerosis data were compared by ANOVA and Tukey tests with significance set at P<0.05.

Fluorescence-activated cell sorting. Hematopoietic- and stromal-enriched BM cells were stained for 20 minutes with FITC-conjugated rat anti-mouse CD45 (leukocyte common antigen, Ly5, clone 30-F11) and phycoerythrin-conjugated rat anti-mouse CD31 (Clone MEC 13.3) antibodies (Pharmingen). Labeled cells were sorted with a dual-laser fluorescence-activated cell sorter (FACS; Becton-Dickinson), and analysis was performed with FlowJo software (version 4.2, Tree Star). Mean results were compared by Student's t test, with significance assumed at P<0.05.

Telomere length assay. DNA (4 to 6 μg) was isolated from cells bluntly scraped from whole aortic intima using DNAzol (Invitrogen). Terminal restriction fragments were prepared and probed as described previously (Gan et al, Pharm. Res. 18:1655-1659 (2001), followed by electrophoretic separation on a 0.3% agarose gel, transfer to filter paper, and phosphorimagery.

ELISA for IL-6. Six-month-old ApoE^{-/-} mice were injected intravenously with 2×10⁶ hematopoietic-enriched BM cells or combined hematopoietic- and stromal-enriched cells from 6-month-old wild type (WT), 6-month-old ApoE^{-/-}, or 4-week-old ApoE^{-/-} donors. Donors were maintained on either regular or high-fat diets. For each donor type, 7 to 8 recipients were treated. At 0, 15, or 30 days after cell injection, plasma interleukin 6 (IL-6) levels were measured by ELISA (R&D Systems).

Results

Age-dependent antiatherosclerotic effect of BM cells. A comparison was made of the efficacy of old versus young donor BM treatment in atherosclerosis prevention in aging ApoE^{-/-} mice. BM cells from severely atherosclerotic, 6-month-old ApoE^{-/-} mice and from recently weaned 4-week-old ApoE^{-/-} mice that had not yet developed detectable atherosclerosis were isolated and cultured. To encompass a host of diverse cell types likely involved in vascular repair (Reyes et al, Blood 98:265-2625 (2001), Sata et al, Nat. Med. 8:403-409 (2002)), the isolated BM was enriched for both hematopoietic and stromal BM fractions. Hematopoietic- and stromal-enriched cells from either young or old ApoE^{-/-} donors were then injected intravenously into unirradiated ApoE^{-/-} recipients maintained on a high-fat diet (1×10 ⁶ cells/injection) every 2 weeks beginning at 3 weeks of age.

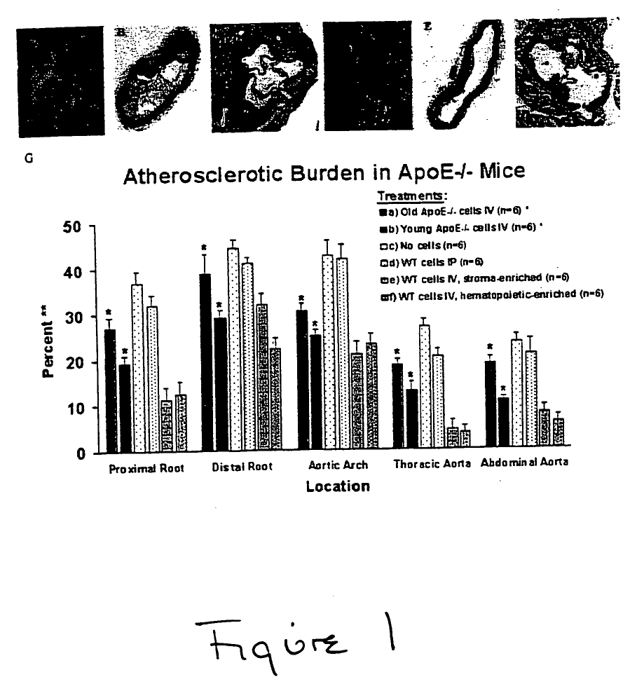

Recipient ApoE^{-/-} mice were killed at 14 weeks of age, after they had received a total of 6 injections. The atherosclerotic burden was determined by 3 complementary techniques: (1) histological analysis of aortic root cross sections, (2) morphometric analysis of transilluminated aortic arches, and (3) en face staining of the aortas with oil red O. Each analysis revealed significantly less atherosclerotic burden in mice that had received combined hematopoietic- and stromal-enriched cells from young ApoE^{-/-} donors (n=6) than in those that had received the same cells from old ApoE^{-/-} donors (n=6; FIGS. 1A through 1F, and FIG. 1G, groups a and b). These findings indicated that (1) aged cells had atherogenic properties, (2) BM-derived cells had atheroprotective properties that were lost with aging and exposure to atherosclerosis, or (3) there was a combination of both processes.

To help address this question, 2 negative controls were used: (1) ApoE^{-/-} mice that received no cells (n=6) and (2) ApoE^{-/-} mice that received 6 injections of young WT BM cells (1×10⁶ cells every 2 weeks, combined hematopoietic enriched and stromal enriched), but this time intraperitoneally (n=6; FIG. 1G, groups c and d). Two positive controls were also used, WT hematopoietic-enriched cells alone and WT stromal-enriched cells alone, each delivered intravenously (FIG. 1G, groups e and f). Whenever male donor BM cells were injected into female recipients, Y-chromosome-positive DNA was consistently detected in the peripheral blood and BM of recipients if the cells were given intravascularly but not if the cells were given intraperitoneally (polymerase chain reaction findings up to 14 days after injection). Levels of atherosclerosis in the negative control groups (FIG. 1G, groups c and d) were similar to the atherosclerotic burden in mice that received old ApoE^{-/-} cells (FIG. 1G, group a). In contrast, mice that received young ApoE^{-/-} or WT cells (FIG. 1G, groups b, e, and f) had less atherosclerotic burden at each anatomic location analyzed. These data indicate that BM cells derived from young, prediseased, animals have an atheroprotective effect, that requires vascular distribution.

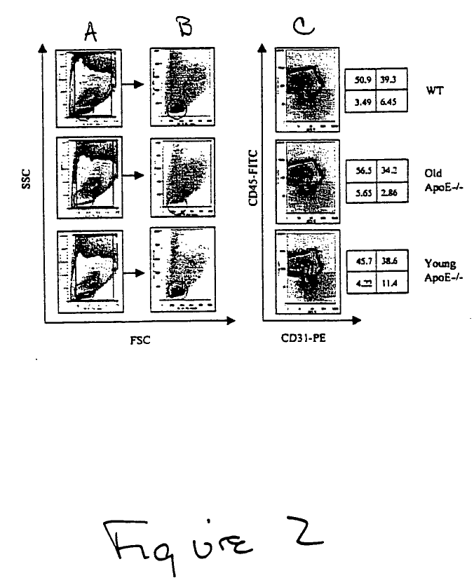

Age-related loss of progenitor cells. The reduced atheroprotective effect of old BM cells suggested that loss of cells with repair capacity might occur with aging. To test this possibility, a study was made of the effect of chronic hypercholesterolemia on BM-cell content. Using FACS, a comparison was made of the percentage of BM cells that expressed established vascular progenitor markers (CD31+/CD45−) in healthy 1-month-old WT mice, young ApoE^{-/-} mice, and 6-month-old ApoE^{-/-} mice with advanced atherosclerosis. FACS revealed that CD31+/CD45− cells (FIG. 2) were significantly diminished in BM from 6-month-old ApoE^{-/-} mice (3.79±2.02% gated cells, n=5) compared with 1-month-old ApoE^{-/-} mice (7.03±2.81% gated cells, n=5) and WT mice (6.36±1.02% gated cells, n=5). This loss of vascular progenitor cells in BM obtained from older ApoE^{-/-} mice may explain, at least in part, the loss of antiatherosclerotic effect of the older ApoE^{-/-} BM cells.

In contrast, FACS analysis of BM from these same groups for the hematopoietic stem cell marker c-kit and the generalized murine stem cell markers sca-1 and CD34 did not reveal any significant deficiencies in old ApoE^{-/-} mice. Furthermore, to discern whether differences in vascular progenitor content might reflect a difference in the vascularity of the BM, FACS analysis was performed for VEGFR-2 (Flk-1), a marker of mature endothelial cells. This analysis revealed an 8.8% (nonsignificant) increase in old ApoE^{-/-} mice relative to young ApoE^{-/-} mice and a slight nonsignificant decrease relative to WT. These data confirm that a specific depletion of intermediate vascular progenitor cells (CD31+/CD45−), without parallel changes in more primitive stem cells (sca-1+, c-kit+, or CD34+) or mature vascular cells (VEGFR-2+), most likely accounted for the age-related loss of BM-derived vascular repair capacity.

To determine whether repeated BM cell injections could replenish the decreased number of CD31+/CD45− cells in aging ApoE^{-/-} mice, FACS was performed for CD31 and CD45 on the recipients' bone marrow. It was found that chronic injection (2 million cells every 1 week for 14 weeks) of combined hematopoietic- and stomal-enriched cells did not significantly restore the deficiency of CD31+/CD45− cells in the BM of aging ApoE^{-/-} mice. The presence of donor cells was, however, detected in the recipient BM by polymerase chain reaction for Y chromosome. These data suggest that rather than reconstituting stem cells in the BM, CD31+/CD45− cells may be actively involved in a vascular repair process with ongoing consumption.

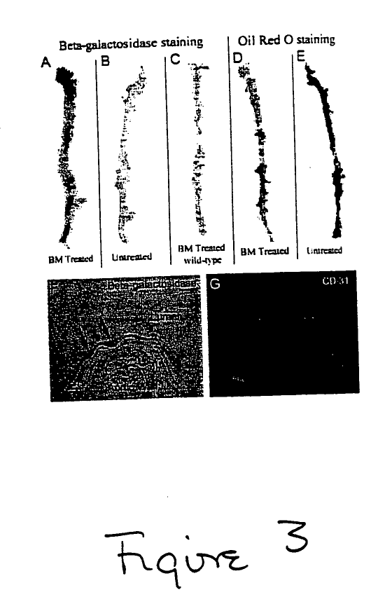

Localization of donor cells. To enable identification of donor-derived cells in recipient mice, combined hematopoietic- and stromal-enriched BM cells from donor mice that expressed β-galactosidase (β-gal) were intravenously injected into ApoE^{-/-} recipients on high-fat diets (n=4) or WT recipients on normal chow diets (n=4) (1×106 cells/injection every 2 weeks for 3 injections). En face aortic β-gal staining in ApoE^{-/-} recipients revealed donor cell localization to the most atherosclerosis-prone regions of the aorta, including the arch, branching points, and distal abdominal region (FIG. 3A). These data, in conjunction with the oil red O staining of paired aortas shown in FIG. 1, revealed significantly less lipid deposition in BM-treated animals (versus untreated), particularly in those regions with the most donor cell engraftment (FIGS. 3A through 3E). Consistent with previous work (Sata et al, Nat. Med. 8:403-409 (2002), histological sections of aortic segments with positive β-gal localization revealed vascular differentiation of donor BM cells (FIG. 3F). β-Gal-positive cells were found to overlie the intima. The predominant phenotype of engrafted cells was endothelial, as demonstrated by colocalization of staining for β-gal and CD31, an endothelium-specific cell marker (FIGS. 3F and 3G). Administration of β-gal-positive BM cells to WT recipients resulted in much fainter en face aortic β-gal staining, with slightly enhanced localization to the arch (FIG. 3C). Untreated ApoE^{-/-} and WT mice had no aortic β-gal staining (FIG. 3B).

Although engrafted cells predominantly expressed CD31, nonendothelial β-gal-positive cells were also observed. A quantitative phenotyping of 220 β-gal-positive cells on aortic histological sections revealed the following: 138 cells (62%) were CD31+/CD45−; 49 (22%) were CD31−/CD45+; 5 (2%) were CD31+/CD45+; and 28 (13%) were CD31−/CD45−. This mixed population of engrafted BM-derived cells might indicate that a variety of cells, including leukocytes, could be involved in vascular repair. As observed previously (Sata et al, Nat. Med. 8:403-409 (2002)), the present data also highlight the possibility that BM-derived cells, when depleted of endothelial progenitors, could instead participate in inflammation and neointima formation. This possibility could theoretically become a more important concern with aging, as the BM becomes exhausted of presumably more salutary CD31+ progenitor cells.

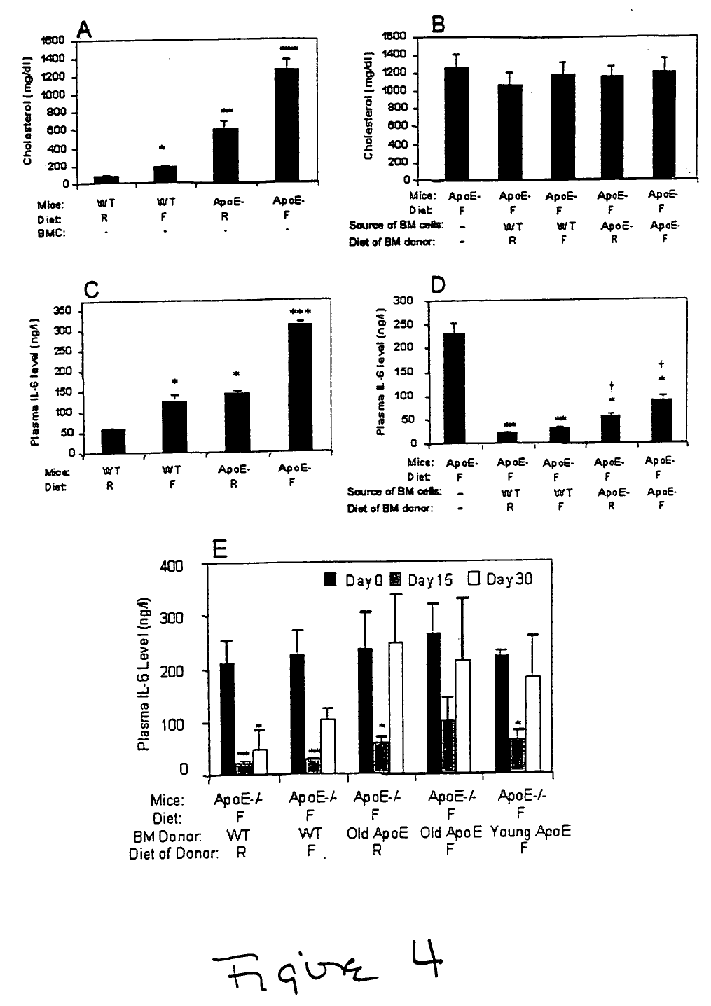

Potential mechanisms of BM-derived atheroprotection. A consideration was made of the potential mechanisms by which injection of young BM-derived cells could delay the progression of atherosclerosis. Attention first turned to cholesterol, the presumed source of atherogenic injury in ApoE^{-/-} mice. It was found that although plasma cholesterol levels varied strikingly with diet and genotype (FIG. 4A), elevated plasma cholesterol levels in ApoE^{-/-} mice (1420±170 mg/dL, n=6, for untreated mice) were not significantly suppressed after injection of any type of BM cells used (eg, 1300±130 mg/dL after 6 injections of WT BM, n=12; additional data for injection of other diet/cell combinations shown in FIG. 4B). These data indicated that the atheroprotective outcome after cell injection was not due to elimination of the hypercholesterolemic source of vascular injury in these mice. The protective mechanism must therefore differ fundamentally from that previously observed in ApoE^{-/-} mice after complete BM ablation and WT reconstitution (Boisvert et al, J. Clin. Invest. 96:1118-1124 (1995), Linton et al, Science 267:1034-1037 (1995)), in which correction of hypercholesterolemia explained, at least in part, the suppression of atherosclerosis.

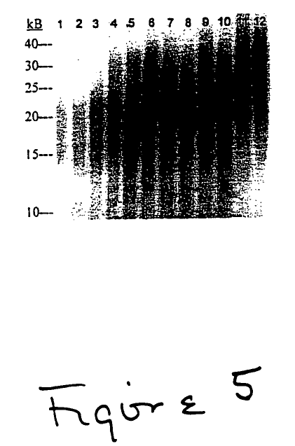

Having observed that BM-derived cells engraft on and “endothelialize” recipient arteries in vivo, attention was turned to the possibility that the cells could locally mediate antiatherosclerotic effects at the level of the arterial wall. One possible mechanism of the antiatherosclerotic impact of engrafted BM cells might be the replacement of senescent endothelial cells by younger cells. Endothelial senescence refers to the acquisition of proinflammatory and proatherosclerotic properties among endothelial cells that have undergone significant telomeric shortening (Minamino et al, Circulation 105:1541-1544 (2002), Chang et al, Proc. Natl. Acad. Sci. USA 92:11190-11194 (1995), Xu et al, FEBS Lett. 470:20-24 (2000), Okuda et al, Atherosclerosis 152:391-398 (2000)). Such shortening is a well-documented and expected consequence of aging (Chang et al, Proc. Natl. Acad. Sci. USA 92:11190-11194 (1995), Xu et al, FEBS Lett. 470:20-24 (2000), Okuda et al, Atherosclerosis 152:391-398 (2000)). Therefore, it was hypothesized that the BM might contain endothelial progenitors that help repair areas of vascular senescence, a function which, if lost with aging and risk factors, would lead to accelerated atherosclerosis. Measurement was made of the average telomere lengths on DNA from cells scraped from the whole aortic intima, comprising not only endothelial but potentially also inflammatory cell DNA. This assessment revealed that ApoE^{-/-} mice had shorter telomeres than healthy age-matched mice (FIG. 5, lanes 1 to 4 versus lanes 11 to 12), whereas the telomeres of intimal cells in ApoE^{-/-} mice that received combined hematopoietic- and stromal-enriched BM cells (1×10⁶ WT cells/injection, every 2 weeks for 6 injections) were significantly longer than those of untreated ApoE^{-/-} mice (FIG. 5, lanes 5 to 10 versus lanes 1 to 4). These data indicate that one potential mechanism of BM-derived vascular repair could be a local effect on reducing endothelial senescence.

Finally, potential humoral effects that could additionally account for the observed amelioration of atherosclerosis were considered. Recent studies indicate that acute phase proteins, produced in response to proinflammatory cytokines such as IL-6, are among the best predictors for severe atherosclerosis and its complications (Taubes et al, Science 296:242-245 (2002), Huber et al, Arterioscler. Thromb. Vasc. Biol. 19:2364-2367 (1999), Ridker et al, Circulation 101:1767-1772 (2000)). Plasma IL-6 levels have been shown to increase with aging and to predict death, fraility, and disability in the elderly (Ferrucci et al, J. Am. Geriatr. Soc. 47:639-646 (1999)). Because the atherosclerotic arterial wall itself produces IL-6, it was hypothesized that BM cell injection could reduce IL-6 production. It was found that plasma IL-6 levels paralleled plasma cholesterol levels in WT/and ApoE^{-/-} mice on regular and fat-rich diets (FIGS. 4A and 4C). However, although injection of BM cells from young ApoE^{-/-} mice (2×10⁶ cells/injection, mixed hematopoietic- and stromal-enriched cells) to ApoE^{-/-} mice on a high-fat diet had no effect on plasma cholesterol levels, it powerfully suppressed the plasma level of IL-6 (FIG. 4D). Instructively, the suppressive effect of BM cells on IL-6 level was significantly weaker when the donor cells originated from 6-month-old ApoE^{-/-} mice, and still weaker if such donors were maintained on a fat-rich diet (FIG. 4D).

Although a precise determination of the molecular mechanism for IL-6 suppression in this model requires additional work, it is likely that the injected cells either suppressed the production of IL-6 by diminishing local and/or systemic vascular inflammation or that a humoral feedback loop was interrupted by cell injection. IL-6 levels fell by a factor of 10 within 15 days after a single cell injection (FIG. 4D). Presumably, this time course is too rapid to have reduced the atherosclerotic burden by such a factor. One explanation is that the persistent elevation of IL-6 and other inflammatory proteins in aging and atherosclerotic disorders could be linked to a lack of BM cells capable of arterial repair. “Injured” blood vessels may trigger the secretion of cytokines, such as IL-6, and growth factors that might help mobilize or “recruit” BM-derived cells for vascular repair. Consistent with this hypothesis are recent findings that circulating levels of endothelial progenitor cells dramatically increase during episodes of active vasculitis (Woywodt et al, Lancet 361:206-210 (2003)), potentially to aid in repair of ongoing vascular damage. The present data indicate that atherosclerosis, and perhaps other chronic inflammatory processes, may, with aging, eventually deplete the BM of progenitor cells. This might then lead to an exhaustion of the vascular repair process, loss of the “negative feedback loop” on IL-6 production, and a consequent heightened cytokine release. This increase in inflammatory cytokines, a signature of advanced atherosclerosis, could then itself participate in further vascular injury and atherosclerosis progression (Taubes et al, Science 296:242-245 (2002), Ridker et al, Circulation 101:1767-1772 (2000)).

Thus, in this mouse model of atherosclerosis, it has been established that there is an atheroprotective property of the BM that is “exhausted” with aging and prolonged exposure to risk factors. Several findings indicate that this exhaustion likely involves progenitor cell-mediated vascular repair. FACS analysis of BM indicated an age-related decline in cells simultaneously expressing endothelial progenitor and lacking leukocyte markers. Moreover, after treatment, more than half of the donor-derived BM cells that engrafted in recipients' arterial vessels exhibited endothelial progenitor characteristics. Although FACS analysis did not reveal quantitative deficiencies of any leukocyte lineages, age-related qualitative or functional differences in leukocytes and other BM-derived cell types may contribute as well. These qualitative traits could include alterations in cholesterol metabolism that do not change the plasma cholesterol, other local biochemical effects on the blood vessel wall, cytokine expression by immune-competent cells, or the acquisition of primed immune cells that exacerbate atherosclerosis. Reduced vascular progenitor content in aging BM could additionally result in a disequilibrium between reparative endothelial cells and inflammatory leukocytes, tipping the balance of injury and repair.

Although it is possible that a single “therapeutic” cell type is exhausted with aging, it appears more plausible that multiple types are affected, each one a component of the vascular repair process. Consistent with previous work (Hill et al, N. Engl. J. Med. 348:593-600 (2003), Sata et al, Nat. Ned. 8:403-409 (2002), Takahashi et al, Nat. Med. 5:434-438 (1999), Lin et al, J. Clin. Invest. 105:71-77 (2000), Reyes et al, J. Clin. Invest. 109:337-346 (2002), Edelberg et al. Circ. Res. 90:e89-e93 (2002)) the present study has identified the apparent importance of CD31+/CD45− cells in vascular repair. However, because of the mixture of cells injected, the role of other cell types cannot be ruled out. Potential confounders could include the effects of self-renewing “true stem cells” or side lineages, such as leukocytes. Much remains to be learned about the repair process and the various cells involved. By optimizing dose and timing of delivery, identifying the cell lineages with the greatest capacity for vascular repair, and eliminating possible proatherosclerotic “contaminant” cells, it is possible that the atheroprotective effects of BM cell injection could be even greater. Identification and restoration of potential age-related qualitative deficiencies in BM cell function could facilitate atheroprotection without the need for actual cell transfer (Goldschmidt-Clermont et al, J. Invasive Cardiol. (Suppl E):18E-26E (2002)).

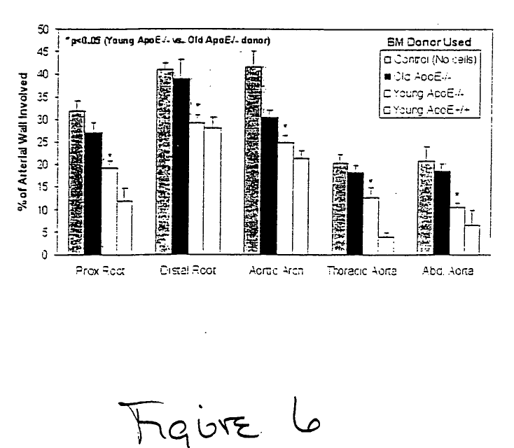

The possibility was tested that with aging, animals that are exposed to such injury exhaust their capacity for vascular cell rejuvenation. Vascular progenitor cell therapy was performed on ApoE/− recipients using BM from either 6-month old atherosclerotic ApoE^{-/-} mice, or from 3-week old ApoE^{-/-} mice, that had not yet developed atherosclerosis. While the older BM reduced atherosclerosis only slightly, BM from young syngeneic ApoE^{-/-} donors had anti-atherosclerotic efficacy approaching that of wild-type C57BL6/J donors (FIG. 6). The lack of therapeutic effect from old BM donors suggests that the vascular progenitor cell content of ApoE^{-/-} BM may diminish with age and may potentially contribute to the development of atherosclerosis in ApoE^{-/-} mice between 3 weeks and 6 months.

All documents cited above are hereby incorporated in their entirety by reference.

What is claimed is:

1. A method of attenuating atherosclerotic progression in a patient comprising administering to said patient progenitor cells in an amount and under conditions such that said attenuation is effected.

2. The method according to claim 1 wherein said cells are endothelial progenitor cells.

3. The method according to claim 1 wherein said cells are pluripotent, bipotent or monopotent stem cells.

4. The method according to claim 1 wherein said cells mature into vascular endothelial cells in said patient.

5. The method according to claim 1 wherein said cells are isolated from an embryo.

6. The method according to claim 1 wherein said cells are isolated from hematopoietic or stromal fractions of bone marrow.

7. The method according to claim 1 wherein said cells are isolated from peripheral blood or umbilical cord blood.

8. The method according to claim 1 wherein said cells are isolated from a non-atherosclerotic mammalian donor.

9. The method according to claim 1 wherein said cells express the CD34+ marker.

10. The method according to claim 1 wherein said cells are heterologous cells.

11. The method according to claim 1 wherein said cells are administered intravenously.

12. The method according to claim 1 wherein said method is used prophylactically.

13. The method according to claim 1 further comprising administering to said patient a proteinaceous or non-proteinaceous anti-atherosclerotic agent.

14. A method of delivering an agent to a vascular site in a patient comprising administering to said patient progenitor cells comprising said agent under conditions such that said delivery is effected.

15. The method according to claim 14 wherein said vascular site is a site of vascular injury.

16. The method according to claim 14 wherein said vascular site is an atherosclerotic site.

17. The method according to claim 14 wherein said agent is a proteinaceous or nonproteinaceous therapeutic agent.

18. The method according to claim 17 wherein said agent is a proteinaceous therapeutic agent.

19. The method according to claim 18 wherein said cells comprise a recombinant molecule comprising a nucleic acid sequence that encodes said proteinaceous agent and, upon administration of said cells, said nucleic acid sequence is expressed and said proteinaceous agent is thereby produced.

20. The method according to claim 19 wherein said nucleic acid sequence is operably linked to a promoter.

21. The method according to claim 20 wherein said promoter is an inducible promoter.

22. The method according to claim 14 wherein said agent is present in a liposome.

23. The method according to claim 14 wherein said agent is an imaging agent.

24. The method according to claim 23 wherein said imaging agent is iron.

25. A method of monitoring cell distribution in a vascular wall of a patient comprising administering to said patient progenitor cells comprising an imaging agent and monitoring distribution of said agent in said vascular wall.

26. The method according to claim 25 wherein said imaging agent is iron.
